(19) United States
(12) Patent Application Publication
(10) Pub. No.: US 2022/0058253 A1
(43) Pub. Date: Feb. 24, 2022

(54) BIOMETRIC AUTHENTICATION APPARATUS

(71) Applicant: ROHM CO., LTD., Kyoto-shi (JP)

(72) Inventors: Hirotoshi USUI, Kyoto-shi (JP); Takenori KATO, Kyoto-shi (JP)

(21) Appl. No.: 17/519,955

(22) Filed: Nov. 5, 2021

Related U.S. Application Data
(63) Continuation of application No. PCT/JP2020/017544, filed on Apr. 23, 2020.

(30) Foreign Application Priority Data
May 7, 2019 (JP) 2019-087721

(51) Int. Cl. G06F 21/32; G01G 19/44

(52) U.S. Cl. CPC G06F 21/32; G01G 19/44

(57) ABSTRACT

A sheet-shaped sensor is laid on the ground on which a person can walk in a barefoot state or a barefoot-equivalent state so as to allow it to come in contact with the soles of the right foot and left foot. A feature value acquisition unit derives a feature value based on a first point positioned in an inner side of a landing portion below a metatarsal bone of the right foot or left foot, a second point positioned in an outer side thereof, and a third point positioned at a landing portion below a calcaneus bone, based on an output of the sheet-shaped sensor generated when the person stands still on the sheet-shaped sensor or when the person is walking. An authentication unit authenticates the person based on the feature value.

### CROSS REFERENCE TO RELATED APPLICATIONS

This application is a continuation under 35 U.S.C. § 120 of PCT/JP2020/017544, filed Apr. 23, 2020, which is incorporated herein reference and which claimed priority to Japanese Application No. 2019-087721, filed May 7, 2019. The present application likewise claims priority under 35 U.S.C. § 119 to Japanese Application No. 2019-087721, filed May 7, 2019, the entire content of which is also incorporated herein by reference.

### BACKGROUND

1. Technical Field

The present disclosure relates to a biometric authentication technique for identifying an individual.

2. Description of the Related Art

Biometric authentication based on human physical characteristics such as fingerprint recognition, iris recognition, finger vein recognition, voiceprint, face shape, handwriting, or the like, has become mainstream.

Such authentication methods have a point in common in that they are performed in a state in which a person is aware of the authentication process. For example, in fingerprint recognition or vein recognition, the person to be authenticated is required to deliberately touch a sensor with his/her finger. In iris recognition, the person to be authenticated is required to fix his/her eye in front of a sensor.

### SUMMARY

The present disclosure has been made in view of such a situation.

An embodiment of the present disclosure relates to a biometric authentication apparatus. The biometric authentication apparatus includes: a sheet-shaped sensor laid on ground on which a person can walk in a barefoot state or a barefoot-equivalent state so as to allow the sheet-shaped sensor to come in contact with soles of left and right feet; a feature value acquisition unit structured to acquire a feature value based on a first point positioned in an inner side of a ground-touching portion below a metatarsal bone of the right foot or the left foot, a second point positioned in an outer side thereof, and a third point positioned at a ground-touching portion below a calcaneus bone, based on an output of the sheet-shaped sensor generated when the person stands still on the sheet-shaped sensor or when the person is walking; and an authentication unit structured to authenticate the person based on the feature value.

Also, the feature value may include coordinate points of the first point through the third point.

Also, the feature value may be described by a triangle defined by the first point through the third point.

Also, the feature value may be generated based on the first point through the third point of the right foot and the first point through the third point of the left foot when the person advances by one step on the sheet-shaped sensor.

Also, the feature value may include the coordinate points of the first point through the third point for each of the right foot and the left foot.

Also, the feature value may be described by a first rectangle with the first point of the right foot and the first point of the left foot as opposite vertices thereof, a second rectangle with the second point of the right foot and the second point of the left foot as opposite vertices thereof, and a third rectangle with the third point of the right foot and the third point of the left foot as opposite vertices thereof.

Also, the feature value may include a vertical length and a horizontal length of each of the first rectangle, the second rectangle, and the third rectangle.

Also, the sheet-shaped sensor may be configured as a pressure sensor. Also, the feature value may include information with respect to a weight measured for at least one from among the first point through the third point.

Also, the feature value acquisition unit may correct a geometric feature value relating to the first point through the third point based on the weight thus measured.

Also, the feature value may include temporal information. Also, the feature value may include temporal information with respect to an interval from a landing of one from among the right foot and the left foot to a landing of the other foot. Also, the feature value may include information with respect to a timing of landing of at least two points from among the first point through the third point.

It should be noted that any combination of the components described above may be made, and a manifestation of the present disclosure may be mutually substituted between a method, apparatus, system, etc., which are also effective as an embodiment of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing a biometric authentication apparatus according to an embodiment;

FIG. 2A is a diagram showing a skeleton of a human foot, and FIG. 2B is a diagram showing a main landing portion of a sole of the foot;

FIGS. 3A through FIG. 3D are diagrams for explaining examples of a feature value;

FIG. 4 is a diagram for explaining the feature value that can be acquired when a person is walking;

FIG. 5 is a diagram showing the feature value acquired with respect to both feet when the person is standing still;

FIG. 6 is a diagram showing the feature value acquired with respect to both feet when the person is walking; and

FIG. 7 is a diagram showing another example of the description of the feature value acquired when the person is walking.

### DETAILED DESCRIPTION

Description will be made below regarding preferred embodiments with reference to the drawings. In each drawing, the same or similar components, members, and processes are denoted by the same reference numerals, and redundant description thereof will be omitted as appropriate. The embodiments have been described for exemplary purposes only, and are by no means intended to restrict the present invention. Also, it is not necessarily essential for the present invention that all the features or a combination thereof be provided as described in the embodiments.

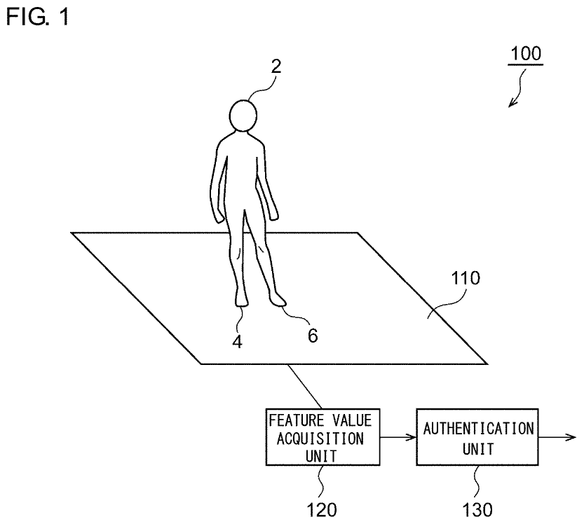

FIG. 1 is a biometric authentication apparatus 100 according to an embodiment. The biometric authentication apparatus 100 includes a sheet-shaped sensor 110, a feature value acquisition unit 120, and an authentication unit 130. The sheet-shaped sensor 110 is laid on the ground or on a floor on which a person 2 can walk in a barefoot state or a barefoot-equivalent state so as to allow the sheet-shaped sensor 110 to come in contact with the sole of the right foot 4 and the sole of the left foot 6. Here, examples of the “barefoot-equivalent state” include a state in which the person wears socks, tabi socks, stockings, or the like. That is to say, the “barefoot-equivalent state” means a state that does not interfere with the acquisition of the foot feature value described later.

The sheet-shaped sensor 110 is configured to be capable of detecting portions pressed by the soles of the feet with strong pressure. As the sheet-shaped sensor 110, a resistor film sensor, capacitive sensor, or the like, may preferably be employed using known techniques. The kind of the sheet-shaped sensor 110 is not restricted in particular.

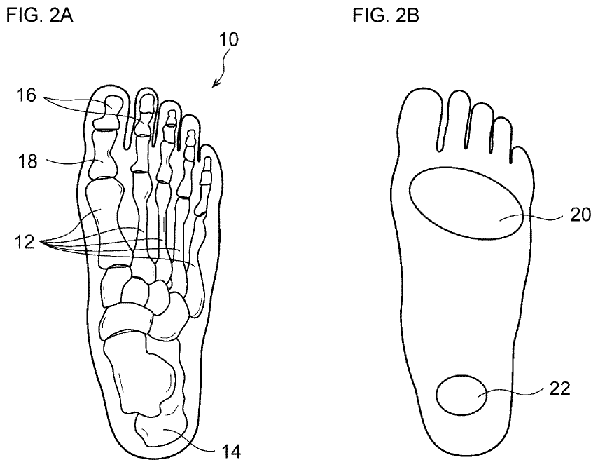

FIG. 2A is a diagram showing a human foot skeleton 10. FIG. 2B is a diagram showing main ground-touching portions of the sole of the foot. As shown in FIG. 2B, typically, the human foot touches the ground at two portions 20 and 22. The ground-touching portion 20 shown in FIG. 2B is positioned below the metatarsal bone 12 shown in FIG. 2A, which will be referred to as a “first ground-touching portion”. On the other hand, the ground-touching portion 22 shown in FIG. 2B is positioned below the calcaneus bone 14 shown in FIG. 2A, which will be referred to as a “second ground-touching portion”.

As a result of investigation by the present inventors, it has been found that, in a standing state or a walking state, the distal phalanges 16 and proximal phalanges 18 move. However, the other bones (joints) do not move. That is to say, it can be assumed that the position relation is substantially unchanging between the first ground-touching portion 20 and the second ground-touching portion 22 in FIG. 2B. In the present embodiment, geometric information with respect to the first ground-touching portion 20 and the second ground-touching portion 22 is used as a biometric authentication feature value.

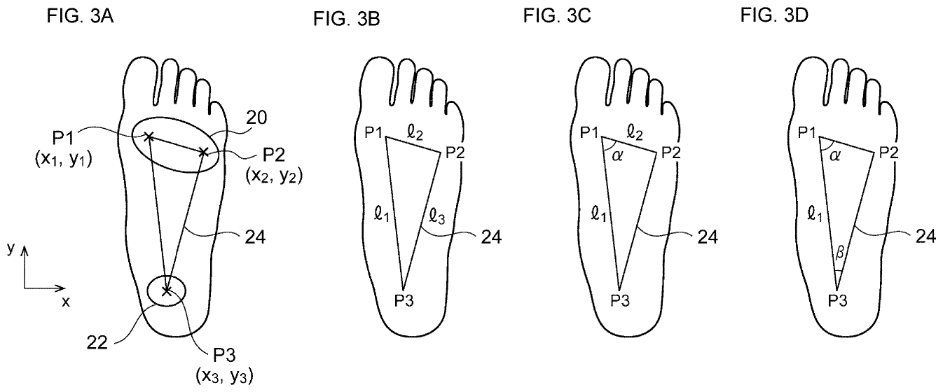

When the person 2 is in a stationary state on the sheet-shaped sensor 110, or when the person 2 walks on the sheet-shaped sensor 110, the feature value acquisition unit 120 shown in FIG. 1 acquires a feature value relating to the geometric information with respect to the first ground-touching portion 20 and the second ground-touching portion 22 shown in FIG. 2B. FIGS. 3A through FIG. 3D are diagrams for explaining an example of the feature value. As shown in FIG. 3A, the first ground-touching portion 20 can be characterized by an inner-side point, i.e., a first point P1, and an outer-side point, i.e., a second point P2. Furthermore, the second ground-touching portion 22 can be characterized by a center point thereof, i.e., a third point P3. The feature value acquisition unit 120 acquires a value with respect to the first point P1, the second point P2, and the third point P3, as a feature value. From another viewpoint, a triangle 24 defined by the first point P1 through the third point P3 is employed as a feature value.

For example, the feature value acquisition unit 120 may employ a combination of the coordinates of each of the first point P1 through the third point P3 as the feature value. Description will be made below with the left-right direction of the person 2 as the x-axis direction, and the traveling direction thereof as the y-axis direction. In this case, the feature value may be represented by P1=(xi, yi), P2=(x2, y2), and P3=(x₃, y₃).

Alternatively, as shown in FIG. 3B, the lengths l₁ through l₃ of the three sides of the triangle 24 defined by the first point P1 through the third point P3 may be employed as the feature value.

Alternatively, as shown in FIG. 3C, a combination of the lengths of two sides l₁ and l₂ from among the three sides of the triangle 24 defined by the first point P1 through the third point P3 and an angle α between the two sides l₁ and l₂ may be employed as the feature value.

Alternatively, as shown in FIG. 3D, a combination of one side l₁ from among the three sides of the triangle 24 defined by the first point P1 through the third point P3 and the angles α and β at both ends of the one side may be employed as the feature value.

Returning to FIG. 1, the authentication unit 130 authenticates the person 2 based on the feature value acquired by the feature value acquisition unit 120. For example, the feature value acquisition unit 120 holds a database in which feature values measured beforehand are registered. The feature value acquisition unit 120 may judge by pattern matching whether or not the currently acquired feature value is registered in the database. The authentication method and the algorithm are not restricted in particular.

The sheet-shaped sensor 110 may be configured as a pressure sensor. In this case, the feature value may include the weight acquired with respect to at least one from among the first point P1 through the third point P3. With this, the feature value includes information with respect to the weight of the person 2 or the like, thereby allowing the authentication accuracy to be improved.

More preferably, the feature value may include the weights applied to the first point P1 and the second point P2. With this, the feature value includes information with respect to the posture of the person 2 (outer-side weighing, inner-side weighing, etc.), thereby providing improved authentication accuracy.

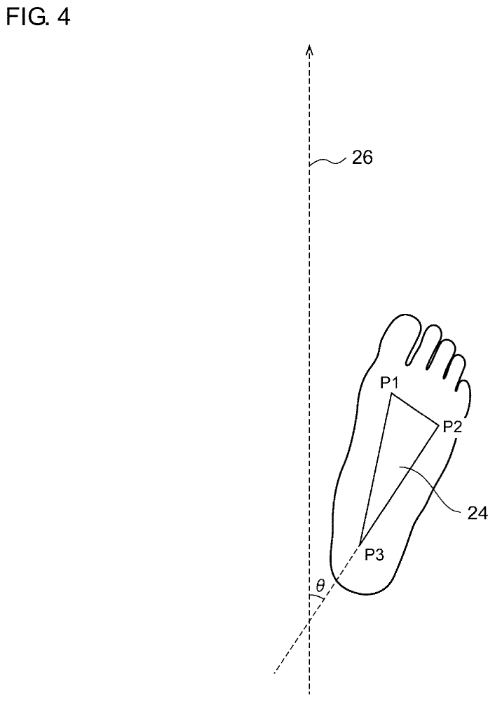

The feature value may more preferably be generated based on the output of the sheet-shaped sensor 110 when the person 2 is walking, rather than when the person 2 is standing still. With such an arrangement in which the feature value is generated when the person 2 is walking, in addition to the information with respect to the triangle 24 defined by the first point P1 through the third point P3, this allows the feature value to include the degree of inclination of the triangle 24 with respect to the traveling direction, i.e., the information with respect to the direction of the foot while walking. FIG. 4 is a diagram for explaining the feature value that can be acquired when the person 2 is walking. For example, the feature value may include a straight line 26 that extends in the traveling direction and an angle θ defined by a line P2-P3.

In the examples shown in FIGS. 3A through FIG. 3D and FIG. 4, the feature value is generated with respect to only one foot (the right foot). Also, the feature value may be acquired for both feet.

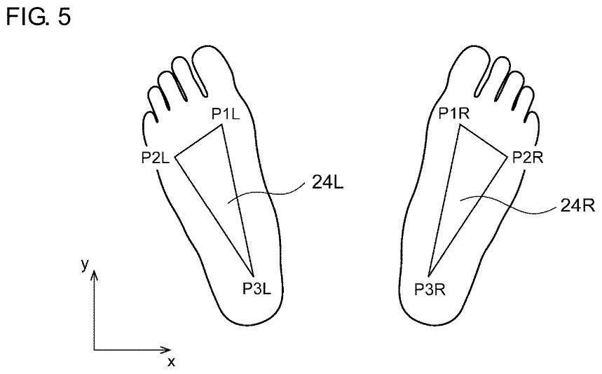

FIG. 5 is a diagram showing the feature value acquired for both feet when the person 2 is standing still. In this case, a triangle 24R (defined by three points P1R through P3R) acquired for the right foot and a triangle 24L (defined by three points P1L through P3L) acquired for the left foot are employed as the feature value. There is a difference in the features of human feet between the left foot and the right foot. Accordingly, such an arrangement provides an increased information amount with respect to the feature value, thereby allowing the authentication accuracy to be improved. In this case, the feature value may also include a relative position relation between the left triangle 24L and the right triangle 24R. For example, the feature value may include the coordinate points of the first point P1L through the third point P3L and the first point P1R through the third point P3R with an arbitrary position as the origin, with the left-right direction of the person 2 as the x-axis direction, and with the traveling direction as the y-axis direction.

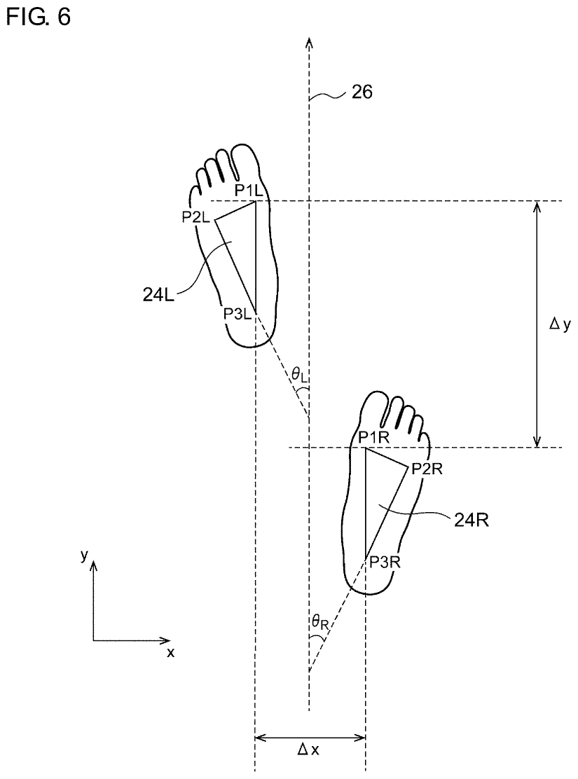

FIG. 6 is a diagram showing the feature value acquired with respect to both feet when the person 2 is walking. The triangle 24R (defined by the three points P1 through P3) acquired for the right foot and the triangle 24L (defined by the three points P1 through P3) acquired for the left foot are employed as basic information of the feature value. In this example, a state is shown in which the left foot is in a forward position. Also, a different state that is the reverse of such a state may be employed. As with a case shown in FIG. 5, there is a difference in the features of human feet between the left foot and the right foot. Accordingly, such an arrangement provides an increased amount of information with respect to the feature value, thereby allowing the authentication accuracy to be improved.

In addition to the information described above, the feature value may include a relative position relation between the left triangle 24L and the right triangle 24R. Also, the feature value may include a desired combination of the stride length Ay, the interval of the left and right feet (stride width) Ax, and the direction of the left foot OL and the direction of the right foot OR. In this case, such an arrangement provides authentication giving consideration to the walking pattern determined by the skeleton of the person 2.

In the case shown in FIG. 6, the coordinate points of the first point P1L through the third point P3L and P1R through P3R may be employed as the feature value with an arbitrary position as the origin, with the left-right direction of the person 2 as the x-axis direction, and with the traveling direction as the y-axis direction.

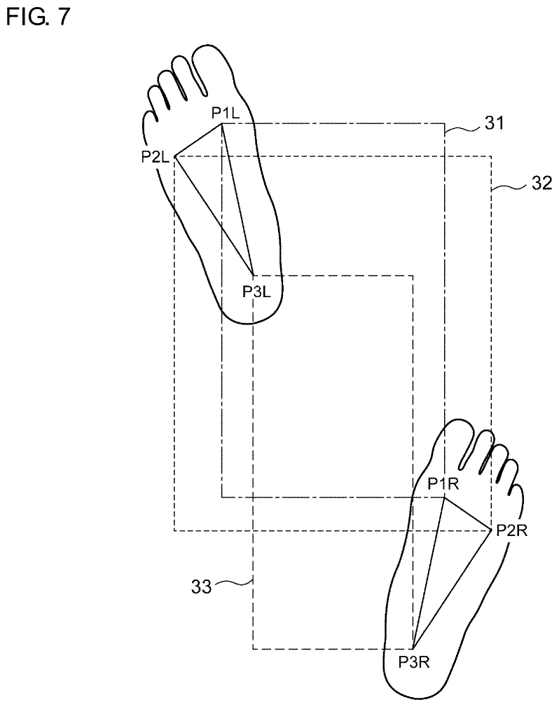

FIG. 7 is a diagram showing another example of the description of the feature value when a person is walking. For example, the feature value may be described based on a first rectangle 31 with the first point P1R of the right foot and the first point P1L of the left foot as its opposite vertices, a second rectangle 32 with the second point P2R of the right foot and the second point P2L of the left foot as its opposite vertices, and a third rectangle 33 with the third point P3R of the right foot and the third point P3L of the left foot as its opposite vertices.

In FIG. 7, the feature value may be described based on the vertical length and the horizontal length of each of the first rectangle 31, the second rectangle 32, and the third rectangle 33.

Description has been made above regarding the present disclosure with reference to the embodiments. The above-described embodiments have been described for exemplary purposes only, and are by no means intended to be interpreted restrictively. Rather, it can be readily conceived by those skilled in this art that various modifications may be made by making various combinations of the aforementioned components or processes, which are also encompassed in the technical scope of the present disclosure. Description will be made below regarding such modifications.

### Modification 1

The feature value may include temporal information. For example, in a case in which the feature value is acquired when a person is walking, the feature value may include the temporal information with respect to an interval from a time point at which the heel lands to a time point at which the midfoot lands.

Alternatively, the feature value may include temporal information with respect to an interval from a time point at which one from among the left foot and the right foot lands to a time point at which the other foot lands.

### Modification 2

In a case of employing the sheet-shaped sensor 110 that is capable of detecting weight, the feature value acquisition unit 120 may correct a geometric feature value (information with respect to the coordinate points, triangles, or rectangles) based on measurement values of the weight. When the person carries and holds heavy baggage, in some cases, this leads to a decrease in the length of stride, an increase in the stride width, or an increase in the angle defined by the left and right feet, resulting in observation of a walking pattern that differs from a normal walking pattern. In order to solve such a problem, judgment may be made based on the measured weight regarding whether or not the person is carrying baggage. When judgement has been made that the person can be assumed to be carrying baggage, the feature value thus measured may be corrected so as to generate a normal feature value for the authentication to be acquired when the person is not carrying baggage. A correction expression to be used in the correction may be generated based on a correspondence relation between the feature values acquired for many people in a state in which they are carrying baggage and a state in which they are not carrying baggage.

### Usage

The usage of the biometric authentication apparatus 100 is not restricted in particular. The biometric authentication apparatus 100 is applicable to authentication in a hospital, security checks in an airport, etc.

Description has been made regarding the present disclosure with reference to the embodiments using specific terms. However, the above-described embodiments show only an aspect of the mechanisms and applications of the present invention. Rather, various modifications and various changes in the layout can be made without departing from the spirit and scope of the present invention defined in appended claims.

What is claimed is:

1. A biometric authentication apparatus comprising:
a sheet-shaped sensor laid on ground on which a person can walk in a barefoot state or a barefoot-equivalent state so as to allow the sheet-shaped sensor to come in contact with soles of left and right feet;
a feature value acquisition unit structured to acquire a feature value based on a first point positioned in an inner side of a ground-touching portion below a metatarsal bone of the right foot or the left foot, a second point positioned in an outer side thereof, and a third point positioned at a ground-touching portion below a calcaneus bone, based on an output of the sheet-shaped sensor generated when the person stands still on the sheet-shaped sensor or when the person is walking; and
an authentication unit structured to authenticate the person based on the feature value.

2. The biometric authentication apparatus according to claim 1, wherein the feature value comprises coordinate points of the first point through the third point.

3. The biometric authentication apparatus according to claim 1, wherein the feature value is described by a triangle defined by the first point through the third point.

4. The biometric authentication apparatus according to claim 1, wherein the feature value is generated based on the first point through the third point of the right foot and the first point through the third point of the left foot when the person advances by one step on the sheet-shaped sensor.

5. The biometric authentication apparatus according to claim 4, wherein the feature value includes the coordinate points of the first point through the third point for each of the right foot and the left foot.

6. The biometric authentication apparatus according to claim 4, wherein the feature value is described by a first rectangle with the first point of the right foot and the first point of the left foot as opposite vertices thereof, a second rectangle with the second point of the right foot and the second point of the left foot as opposite vertices thereof, and a third rectangle with the third point of the right foot and the third point of the left foot as opposite vertices thereof.

7. The biometric authentication apparatus according to claim 6, wherein the feature value includes a vertical length and a horizontal length of each of the first rectangle, the second rectangle, and the third rectangle.

8. The biometric authentication apparatus according to claim 1, wherein the sheet-shaped sensor is configured as a pressure sensor.

9. The biometric authentication apparatus according to claim 8, wherein the feature value includes information with respect to a weight measured for at least one from among the first point through the third point.

10. The biometric authentication apparatus according to claim 8, wherein the feature value acquisition unit corrects a geometric feature value relating to the first point through the third point based on the weight thus measured.

11. The biometric authentication apparatus according to claim 1, wherein the feature value includes temporal information.

12. The biometric authentication apparatus according to claim 11, wherein the feature value includes temporal information with respect to an interval from a landing of one from among the right foot and the left foot to a landing of the other foot.

13. The biometric authentication apparatus according to claim 11, wherein the feature value includes information with respect to a timing of landing of at least two points from among the first point through the third point.
